(19) United States
(12) Plant Patent Application Publication
(10) Pub. No.: US 2012/0180169 P1
(43) Pub. Date: Jul. 12, 2012

(54) Shrub rose plant named 'Poulren019'

(50) ;

(76) Inventor: Mogens Nyegaard Olesen, Fredensborg (DK)

(21) Appl. No.: 12/930,440

(22) Filed: Jan. 7, 2011

(51) Int. Cl. A01H 5/00

(52) U.S. Cl. PLT/107

(57) ABSTRACT

A new garden rose plant of the shrub class which has abundant, deep pink, fragrant flowers, and attractive foliage. This new and distinct variety has shown to be uniform and stable in the resulting generations from asexual propagation.

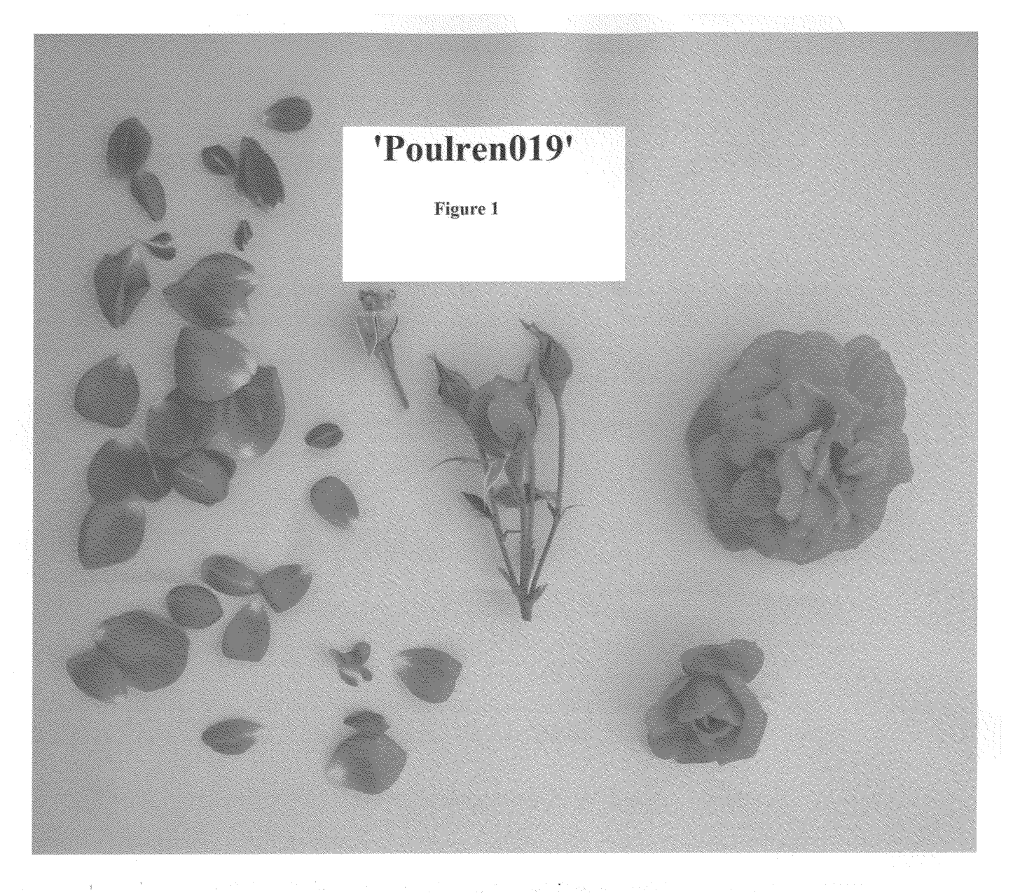

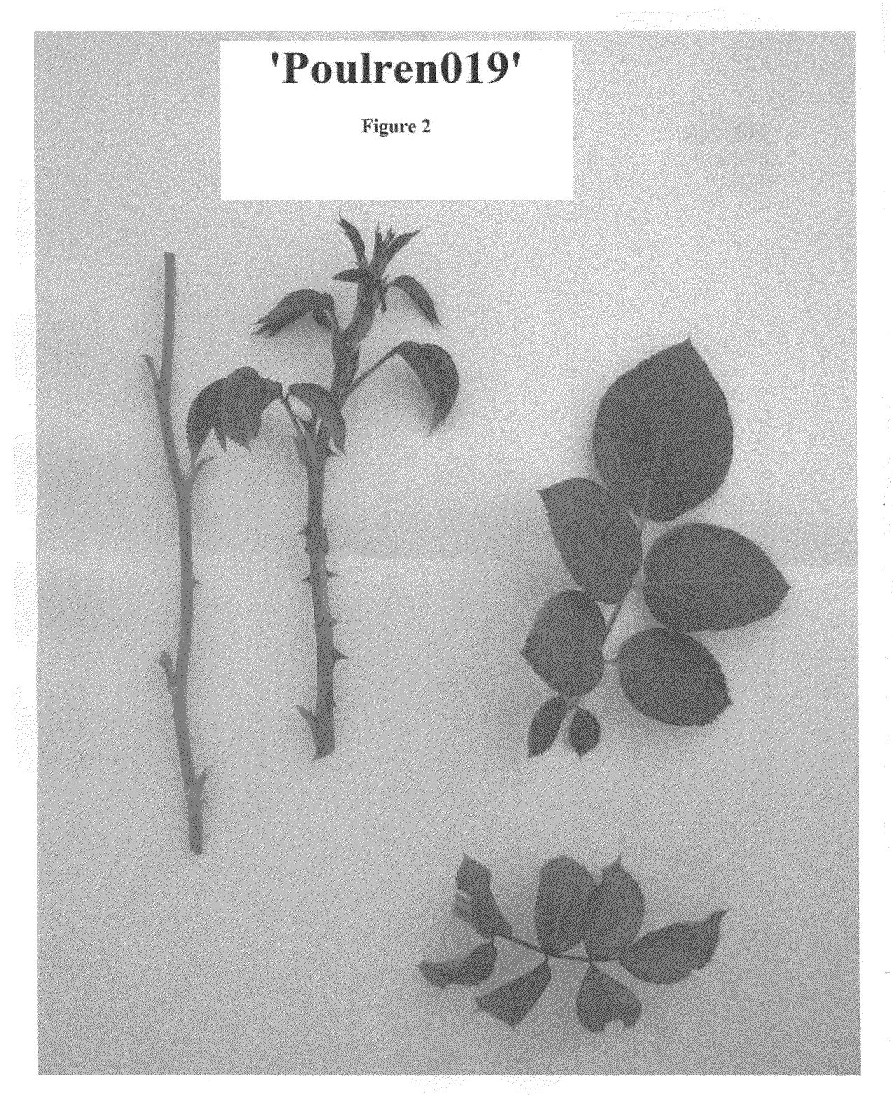

### BOTANICAL DESIGNATION

Rosa hybrid

### VARIETY DENOMINATION

‘Poulren019’

### SUMMARY OF THE INVENTION

The present invention constitutes a new and distinct variety of garden rose plant which originated from a controlled crossing between the female seed parent, an unnamed, non-commercial seedling, and the male pollen parent, also an unnamed, non-commercial seedling.

The two parents were crossed during the summer of 2001 and the resulting seeds were planted in a controlled environment in Fredensborg, Denmark. The new variety, named ‘Poulren019’, originated as a single seedling from the stated cross.

The new variety may be distinguished from its female seed parent by the following combination of characteristics:

1. The seed parent has smaller flowers than ‘Poulren019’.

2. The seed parent has light green foliage while the foliage of ‘Poulren019’ is dark green.

‘Poulren019’ has more abundant flowers than the male pollen parent.

The objective of the hybridization of this rose variety was to create a new and distinct variety for garden use with unique qualities, such as:

1. Uniform and abundant red-purple flowers;

2. Vigorous, but compact growth when propagated both as a budded rose and on its own roots;

3. Exceptional disease resistance;

4. Fragrant flowers.

This combination of qualities is not present in previously available commercial cultivars of this type, known to the inventor, and distinguish ‘Poulren019’ from all other varieties of which we are aware.

As part of the rose development program, Mogens N. Olesen germinated the seeds from the aforementioned hybridization during winter of 2001 and conducted evaluations on the resulting seedlings in a controlled environment in Fredensborg, Denmark. ‘Poulren019’ was selected in the spring of 2002 by the inventor as a single plant from the progeny of the aforementioned hybridization.

Asexual reproduction of ‘Poulren019’ by traditional budding and rooted cuttings was first done by Mogens N. Olesen in the nursery in Fredensborg, Denmark in July, 2002. This initial and other subsequent asexual propagations conducted in controlled environments have demonstrated that the characteristics of ‘Poulren019’ are true to type and are transmitted from one generation to the next.

### BRIEF DESCRIPTION OF THE DRAWING

The accompanying color illustrations show as true as is reasonably possible to obtain in color photographs of this type, the typical characteristics of the buds, flowers, leaves, and stems, of ‘Poulren019’. Specifically illustrated in the drawings are: open flowers, flower petals, reproductive parts, stems, and leaves showing juvenile and mature growth.

### DETAILED DESCRIPTION OF THE VARIETY

The following is a description of ‘Poulren019’, as observed in its growth in in a field nursery in Jackson County, Oregon. Observed plants are 3 years of age, and were grown on Rosa multiflora understock. Color references are made using The Royal Horticultural Society (London, England) Colour Chart, 2001, except where common terms of color are used.

For a comparison, several physical characteristics of the rose variety ‘Pouldra’, U.S. Plant Pat. No. 12,999 are compared to ‘Poulren019’ in Chart 1.

CHART 1

| | | |
|---|---|---|
| | ‘Poulren019’ | ‘Pouldra’ |
| Petal Count | 75 to100 petals | 20 to 30 petals. |
| Flower Diameter | 60 to 80 mm | 100 mm |
| Genaral Tonality | Red-Purple Group N57B to | Red-Purple Group 57B |
| of Flower Color | Red-Purple Group 57C | |

Flower and flower bud:
   Blooming habit.—Repeat flowering.
   Flower bud.—Size: Upon opening, 20 to 25 mm in length from base of receptacle to end of bud. Bud diameter is 11 mm. Bud form: Ovoid. Bud color: As sepals unfold, petals are Red-Purple Group 61 A. Sepal inner surface: Color: Yellow-Green Group 147C. Surface: Moderate pubescence observed. Sepal outer surface: Color: Yellow-Green Group 146B with moderate anthocyanic pigments the color of Greyed-Purple Group 187B observed. Texture: Smooth. Sepal Shape: Apex: Cirrhose. Base: Flat at union with receptacle. Sepal Margin: Margins have medium foliaceous appendages on three of the five sepals. Sepal size: Average 30 mm long by 8 mm wide. Receptacle: Texture: Smooth. Shape: Campanulate. Size: 5 mm tall by 6 mm wide. Color: Yellow-Green Group 146C. Peduncle: Length: 10 to 15 cm. Diameter: 2 mm. Color: Yellow-Green Group 146B Pedicel: Surface: Smooth. Length: 45 mm on average. Diameter: 2 to 3 mm. Color: Yellow-Green Group 146C. Strength: Moderate.
   Flower bud development.—In clusters of 3 to 5 flower buds per stem.
   Flower bloom.—Fragrance: Strong perfume. Duration: As a cut flower 5 to 8 days. The blooms have a duration on the plant of approximately 8 to 10 days. Petals fall cleanly away from plant after flowers have fully matured. Size: Flower diameter is 60 to 80 mm when open. Flower depth is 25 to 30 mm. Flower shape: As the flower opens, the form is cupped, with many overlapping petals packed vertically into quarter sections. Opening fully, the cup shape expands to become an opened rosette. Shape of flower, side view: Upon opening the upper portion is flat. The lower portion is concave. After opening, the upper portion is convex, while the lower portion is concave.
   Petalage.—Under normal conditions, flowers have 75 to 100 petals total, 10 to 15 of which are petaloids.
   Petal color.—Upon opening, outer petals Upper surface: Red-Purple Group N57B. Lower surface: Red-Purple Group N57C. Upon opening, inner petals: Upper surface: Red-Purple Group N57A. Lower surface: Red-Purple Group N57B. Basal petal spots, upon opening: Upper surface: Yellow Group 5B. Lower surface: Yellow Group 5 B to 5C. After opening, outer petals Upper surface: Red-Purple Group N57C. Lower surface: Red-Purple Group N57D. Basal petal spots, after opening: Upper and Lower: Green-White Group 157B.
   General tonality.—Newly opened flowers are Red-Purple Group N57B to 57C. After the flowers have opened for 5 days, general tonality is Red-Purple Group N57C.
   Petals.—Petal Reflex: None. Margin: Entire and uniform. Shape: Generally narrow elliptic. Apex shape: Rounded. Base shape: Varying from acute to rounded. Size: Approximately 50% of the petals are in the small range, 20 to 30 mm long by 12 to 15 mm wide. The remainder of the petals are in the large size range of mm (1)×35 mm (w). Texture: Smooth. Thickness: Average.
   Petaloids.—Quantity: 10 to 15. Shape: Elliptic, with an acute apex and base. Color: Upper surface is Red-Purple Group N57A. Lower surface is Red-Purple Group N57B. Basal petal spots are Yellow Group 5B. Size: 12 mm long by 5 mm wide.
   Reproductive organs.—Pollen: None observed. Anthers: Size: 7 to 8 mm in length. Color: Yellow Group 13B. Quantity: 50 on average. Filaments: Color: Yellow Group 4B. Length: 3 mm. Pistils: Length: 9 mm. Quantity: 35 on average. Stigmas: Superior to the height of filaments and anthers. Color: Yellow Group 11D. Styles: Color: Yellow-White Group 158D with intonations of Red Group 43D. Hips: None Observed in the field nursery in Jackson County Oregon.
Plant:
   Plant growth.—Upright to bushy. When grown as a budded field grown plant on Rosa multiflora understock, the average height of the plant is 150 cm and the average width is 130 cm.
   Stems.—Color: Juvenile growth: Yellow-Green Group 14C, with anthocyanic intonations of Greyed-Purple Group 183C. Mature growth: Yellow-Green Group 146B. Length: On average, canes are 35 cm from the base of the plant to the flowering portion. Diameter: 5 to 7 mm. Internodes: On mature canes, there is an average distance of 50 mm between nodes. Surface Texture: Young wood: Smooth. Older wood: Smooth.
   Prickles.—Incidence: 4 prickles per 10 cm of stem. Size: Average length of prickles on mature stems is 5 mm. Shape: Linear on upper portion, and deep concave underneath. Color: Juvenile prickles: Greyed-Purple Group 185A. Mature prickles: Greyed-Purple Group 184A with intonations of Greyed-Orange Group 164C.
   Plant foliage.—Normal number of leaflets leaves in middle of the stem: 7 leaflets. Compound leaf: 140 to 150 mm (1)×100 to 110 (w). Quantity: 3 leaves per 10 cm of stem on average. Color of mature foliage: Upper side: Yellow-Green Group 147A. Lower side: Yellow-Green Group 147C. Color of juvenile foliage: Upper side: Yellow-Green Group 146A. Lower side: Yellow-Green Group 146B. Anthocyanin: Greyed-Purple Group 183A completely saturating newest leaves.
   Plant leaves and leaflets.—Stipules: Size: 19 mm in length. Quantity: 2 per compound leaf. Shape: Linear, slightly broad based with outward extending apices. Margins: Finely serrated with many stipitate glands. Color: Yellow-Green Group 146B. Petiole: Length: 20 mm. Diameter: 2 mm. Upper surface: Color: Yellow-Green Group 146A with intonations of Greyed-Red Group 178A. Lower surface: Color: Yellow-Green Group 144B. Observations: Few stipitate glands observed. Rachis: Length: 50 to 70 mm. Color: Yellow-Green Group 146A with intonations of Greyed-Red Group 178A. Lower surface: Color: Yellow-Green Group 144B. Observations: Few stipitate glands observed. Leaflet: Edge: Serrated. Size: The terminal leaflet on normal leaves is 50 to 65 mm in length by 40 to 55 mm wide. Shape: Generally elliptical. Base: Cordate. Apex: Mucronate Texture: Smooth. Thickness: Thick. Arrangement: Odd pinnate. Venation: Reticulate. Glossiness: Moderately glossy.
   Disease resistance.—Above average resistance to powdery and downy mildew, rust, black spot, and Botrytis under normal growing conditions in Jackson County, Oregon.
   Cold hardiness.—The variety is tolerant to USDA Cold Hardiness Zone 6.
   Heat tolerance.—The variety has been found to be suitable for climate conditions found in the American Horticulture Society heat zone 7.

1. A new and distinct variety of rose plant of the shrub rose class named ‘Poulren019’, substantially as illustrated and described herein, due to its abundant deep pink flowers, disease resistance, and extended period of bloom.
